(19) United States
(12) Patent Application Publication
(10) Pub. No.: US 2008/0171978 A1
(43) Pub. Date: Jul. 17, 2008

(54) Balloon in balloon chemotherapy catheter and method

(75) Inventor: Matthew Richard Quigley, Pittsburgh, PA (US)

Correspondence Address: Ansel M. Schwartz;Attorney at Law, Suite 304, 201 N. Craig Street, Pittsburgh, PA, 15213, US

(73) Assignee: Allegheny-Singer Research Institute

(21) Appl. No.: 11/653,032

(22) Filed: Jan. 12, 2007

(51) Int. Cl. A61M 25/10

(52) U.S. Cl. 604/101.02

(57) ABSTRACT

An apparatus for administering a drug to a tumor of a patient includes a first balloon made of a semi-permeable material in which the drug is disposed. The apparatus includes a second balloon disposed in the first balloon, which when expanded forces the drug out of the first balloon, the first and second balloons adapted to be positioned in the tumor for a predetermined post-operative period. A method for administering a drug to a tumor of the patient includes the steps of placing a first balloon having a second balloon disposed in the first balloon in the tumor in the patient. There is the step of filling the first balloon with the drug. There is the step of filling the second balloon with a fluid to expand the second balloon and cause the drug in the first balloon this to be driven out of the first balloon into the tumor.

### FIELD OF THE INVENTION

The present invention is related to chemotherapy using a balloon in balloon catheter. More specifically, the present invention is related to chemotherapy using a balloon in balloon catheter including a double lumen catheter with a first port connected with the first balloon and a second port connected with the second balloon.

### BACKGROUND OF THE INVENTION

In tumor treatment, there is a need to provide drug therapy directly to the tumor over long periods of time. Gliadel is an FDA-approved implantable wafer form of BCNU chemotherapy left in the resection bed following removal of malignant primary tumors. These wafers leech chemotherapy for 7-30 days into the surrounding tissue and have been shown to modestly, but statistically significantly, prolong survival. The other method consists of an array of thin catheters implanted percutaneously connected to a pump which slowly delivers a small volume of drug over a prolonged period of time. Disadvantages are the separate procedure for implantation of catheters, hospitalization during infusion and removal of the catheters following a single round of therapy. Efficacy has not been proven. The device is non-proprietary.

### BRIEF SUMMARY OF THE INVENTION

The present invention pertains to an apparatus for administering a drug to a tumor of a patient. The apparatus comprises a first balloon made of a semi-permeable material in which the drug is disposed. The apparatus comprises a second balloon disposed in the first balloon, which when expanded forces the drug out of the first balloon, the first and second balloons adapted to be positioned in the tumor for a predetermined post-operative period.

The present invention pertains to a method for administering a drug to a tumor of the patient. The method comprises the steps of placing a first balloon having a second balloon disposed in the first balloon in the tumor in the patient. There is the step of filling the first balloon with the drug. There is the step of filling the second balloon with a fluid to expand the second balloon and cause the drug in the first balloon this to be driven out of the first balloon into the tumor.

### BRIEF DESCRIPTION OF THE SEVERAL VIEWS OF THE DRAWING

In the accompanying drawings, the preferred embodiment of the invention and preferred methods of practicing the invention are illustrated in which:

FIG. 1 is a schematic representation of the apparatus of the present invention.

### DETAILED DESCRIPTION OF THE INVENTION

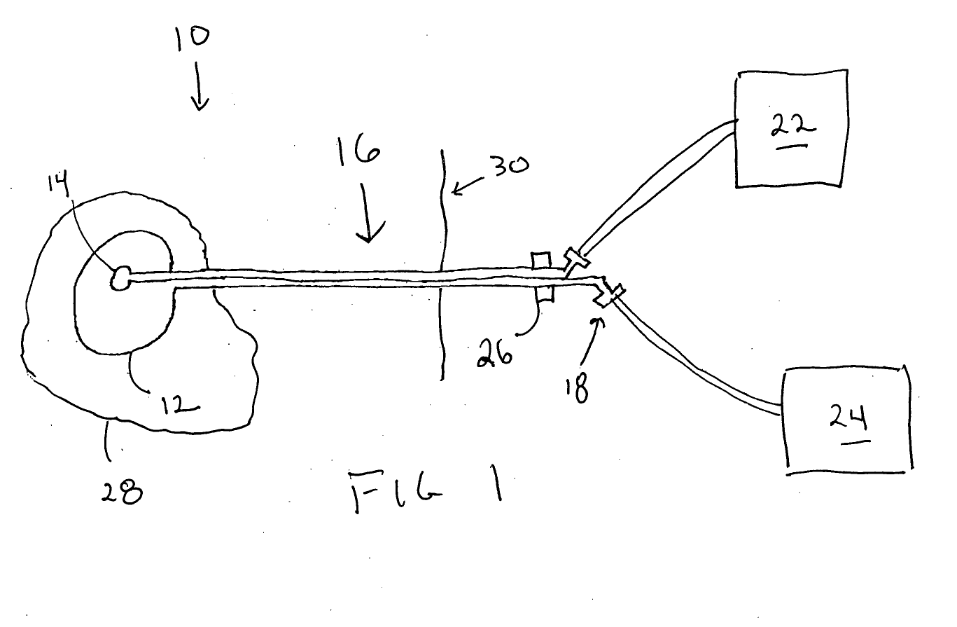

Referring now to the drawings wherein like reference numerals refer to similar or identical parts throughout the several views, and more specifically to FIG. 1 thereof, there is shown an apparatus 10 for administering a drug to a tumor 28 of a patient 30. The apparatus 10 comprises a first balloon 12 made of a semi-permeable material in which the drug is disposed. The apparatus 10 comprises a second balloon 14 disposed in the first balloon 12, which when expanded forces the drug out of the first balloon 12, the first and second balloons 12, 14 adapted to be positioned in the tumor 28 for a predetermined post-operative period.

Preferably, the apparatus 10 includes a double lumen catheter 16 with a first port 18 connected with the first balloon 12 and a second port 20 connected with the second balloon 14. The apparatus 10 includes a fluid source 22 connected to the second port 20 to provide fluid to the second balloon 14 to expand the second balloon 14. Preferably, the apparatus 10 includes a drug source 24 connected to the first port 18 to provide the drug to the first balloon 12. The fluid source 22 can be filled with saline to expand the second balloon, or any other benign fluid to a patient.

The present invention pertains to a method for administering a drug to a tumor 28 of the patient 30. The method comprises the steps of placing a first balloon 12 having a second balloon 14 disposed in the first balloon 12 in the tumor 28 in the patient 30. There is the step of filling the first balloon 12 with the drug. There is the step of filling the second balloon 14 with a fluid to expand the second balloon 14 and cause the drug in the first balloon 12 this to be driven out of the first balloon 12 into the tumor 28. Preferably, there is the step of pulling the first and second balloons 12, 14 out of the patient 30 after a predetermined period of at least one day has passed after the placing step.

In the operation of the invention, the apparatus 10 is implanted at the time of surgical excision of a tumor 28 mass which is sized appropriately to the cavity for the purpose of local delivery of an active drug such as chemotherapy during the extended post-operative period. The implant consists of a double-lumen catheter 16 with two ports for separate access to each of two balloons which are placed inside the resection cavity. The balloons are fashioned one inside the other. The inner balloon is made of a non-permeable material; the outer is of a semi-permeable material. Filling the inner balloon conforms the device to the shape of the resection bed and drives the contents of the outer balloon into the bed itself. The other end of the catheter 16 contains ports for access to the lumens and would reside outside the body for easy and frequent access. When no longer required, the device can be explanted by simply “pulling” it from the port site.

The apparatus 10 provides high doses of chemotherapy to the surgical resection bed in any solid tumor 28. Local delivery allows for the application of supra-therapeutic drug levels with minimal systemic absorption, thus minimizing morbidity of treatment. The apparatus 10 is implanted at the time of the primary surgery and does not require a separate procedure. The chemotherapy delivered can be altered by agent and dose. Treatments can be performed in an outpatient 30 setting and repeated over the life of the device which could be estimated at 6-12 months. One example of a drug for use is BCNU.

The first balloon 12 is made of latex or any semi-permeable material benign to patients 30. The second balloon 14 is made of latex or any non-permeable material benign to patients 30. A valve 26 to control flow to the first and second balloons 12, 14 is similar to that used in common devices such as a foley catheter 16. The rate of flow of drug into the patient 30 from the first balloon 12 would be up to 5 cc/day.

Although the invention has been described in detail in the foregoing embodiments for the purpose of illustration, it is to be understood that such detail is solely for that purpose and that variations can be made therein by those skilled in the art without departing from the spirit and scope of the invention except as it may be described by the following claims.

1. An apparatus for administering a drug to a tumor of a patient comprising:
a first balloon made of a semi-permeable material in which the drug is disposed; and
a second balloon disposed in the first balloon, which when expanded forces the drug out of the first balloon, the first and second balloons adapted to be positioned in the tumor for a predetermined post-operative period.

2. An apparatus as described in claim 1 including a double lumen catheter with a first port connected with the first balloon and a second port connected with the second balloon.

3. An apparatus as described in claim 2 including a fluid source connected to the second port to provide fluid to the second balloon to expand the second balloon.

4. An apparatus as described in claim 3 including a drug source connected to the first port to provide the drug to the first balloon.

5. A method for administering a drug to a tumor of the patient comprising the steps of:
placing a first balloon having a second balloon disposed in the first balloon in the tumor in the patient;
filling the first balloon with the drug; and
filling the second balloon with a fluid to expand the second balloon and cause the drug in the first balloon this to be driven out of the first balloon into the tumor.

6. A method as described in claim 5 including the step of pulling the first and second balloons out of the patient after a predetermined period of at least one day has passed after the placing step.
